(19) United States
(12) Patent Application Publication
(10) Pub. No.: US 2025/0221862 A1
(43) Pub. Date: Jul. 10, 2025

(54) COMPRESSIVE HEAT EXCHANGER

(71) Applicant: WEBER STATE UNIVERSITY, Ogden, UT (US)

(72) Inventors: Valerie Wirth HERZOG, Ogden, UT (US); Taylor FOSS, Ogden, UT (US)

(21) Appl. No.: 19/013,866

(22) Filed: Jan. 8, 2025

Related U.S. Application Data
(60) Provisional application No. 63/618,460, filed on Jan. 8, 2024.

(51) Int. Cl. A61F 13/00

(52) U.S. Cl. CPC A61F 13/00987; A61F 2013/00187; A61F 2013/00319

(57) ABSTRACT

The present disclosure is directed toward heat exchangers, and more particularly to an elastic bandage with a non-adhering film. The clastic bandage may be submerged in a biocompatible liquid, frozen, unwound, and then compressively wrapped around a complex shape such as a portion of a human body and easily unwrapped after usage.

### CROSS-REFERENCES TO RELATED APPLICATIONS

This application claims priority to and the benefit of U.S. Provisional Patent Application No. 63/618,460, filed Jan. 8, 2024, the entirety of which is incorporated herein by this reference.

### BACKGROUND

The present application is directed toward heat exchangers and more particularly to an elastic bandage with a non-adhering film which elastic bandage may be compressively wrapped around a complex shape such as a portion of a human body and easily unwrapped after freezing and usage.

Heat exchangers are used in therapeutic applications to regulate body temperature for specific medical benefits. Lowering body temperature helps reduce inflammation, alleviate pain, and minimize secondary metabolic injury. This occurs because cooling slows down cellular activity and metabolic processes, thereby limiting further tissue damage. It also causes vasoconstriction, which reduces blood flow to the affected area, thereby minimizing swelling and fluid buildup. These effects are particularly valuable in treatments like cryotherapy for managing acute injuries, post-surgical recovery, and chronic pain conditions. The heat exchangers function by having a material with a lower temperature than the body that absorbs the heat from the body and thereby lowers the body's temperature. Heat exchangers may be passive or active.

Active heat exchangers typically include a liquid medium, such as water, that circulates through channels in a panel. The panel cools the body by transferring heat to the liquid medium, which is then cycled through ingress and egress tubes connected to a cooling tank. The cooling tank often contains ice water and is equipped with a pump to circulate the liquid. While effective, active heat exchangers tend to be expensive, cumbersome, and difficult to transport. They also require regular maintenance, such as refilling the cooling tank with liquid and ice, which can be inconvenient. Active heat exchangers may also cause excessive cooling, causing tissue damage if left on for an extended period of time.

In contrast, passive heat exchangers are simpler and more cost-effective. These may include ice packs or cooling packs that are placed directly on the body. Common passive cooling packs include a fabric cover that houses a gel packet or liquid medium. Some fabric covers used in these systems incorporate adhering fabric with hooks made of nylon or other fabric polymers known in the art. These hooks allow the pack to attach securely to the body or other surfaces, enhancing usability. The gel packets often consist of water mixed with chemical agents such as ammonium nitrate, calcium ammonium nitrate, or urea, which activate via an endothermic reaction when pressure is applied. Liquid mediums in passive cooling packs can include substances like diethylene glycol, propylene glycol, ethylene glycol, or solutions thereof. These cooling packs may also incorporate thickening agents, such as vinyl-coated silica gel, hydroxyethyl cellulose, or sodium polyacrylate gel beads, and are often dyed with non-toxic blue coloring for easy identification. The temperature of these materials can be lowered in a freezing apparatus before use. Passive heat exchangers are more portable and easier to use than active systems. However, they often struggle to conform tightly to the body, especially around complex shapes like joints, limiting their effectiveness in delivering consistent cooling and compression.

Both passive and active heat exchangers commonly used in the art have limitations in their ability to conform and adhere tightly to a body part, particularly in areas with complex shapes like joints. Insufficient adhesion reduces the efficiency of cooling and compression therapy. To address these limitations, affordable and innovative solutions are needed to provide effective cooling and compression while overcoming challenges like poor adhesion, excessive cooling, or difficulty in application and reusability.

### SUMMARY

One or more embodiments of the present disclosure provide a heat exchanger made from a conformable elastic bandage, which includes an elastomeric substrate with opposing surfaces, and a non-adhering film of temperature-resistant, biocompatible material applied to at least a portion of one surface of the substrate.

The non-adhering film may be made from a hydrophobic film. Said hydrophobic films can be made from temperature resistant polyethylene, polyvinylidene chloride, polyurethane, and silicones. In preferred embodiments, the non-adhering film can include pure silicone, entirely coating one side of the entire elastic bandage.

An embodiment of the elastic bandage can be produced through a long flat strip of elastomeric substrate that is used to support and compress a complex shape. In preferred embodiments, said elastic bandage may have various widths and lengths. Another embodiment of the elastic bandage can be produced through a long tubular strip of elastomeric substrate.

Said elastomeric substrate may preferably be comprised of a combination of synthetic and natural fibers. The synthetic and natural fibers may be spun into thread which may be woven or nonwoven, such as through meltblown extrusion, to make the elastomeric substrate.

In preferred embodiments, said elastic bandage can be applied to a user to provide varying levels of compressive force (e.g., between 1 to 70 mmHg) to suit different therapeutic needs, such as reducing swelling or improving circulation. The bandage preferably exhibits high stretchability, making it adaptable and easy to wrap around various body parts. In preferred embodiments, the bandage also exhibits low hysteresis, enabling it to retain its elasticity overtime despite repeated usage.

In exemplary embodiments, the elastomeric substrate of said elastic bandage can be sufficiently hydrophilic to absorb biocompatible liquids. Hydrophobic polymers, which naturally resist water absorption, can be treated with hydrophilic coatings to modify their surface properties. These biocompatible liquids may be used by themselves, in combination, or as solvents in solution with biocompatible solutes. Preferably the biocompatible liquids have a freezing point sufficiently low to provide a cryotherapeutic effect when applied to a body part.

The non-adhering film can be adhered to the elastomeric substrate through extrusion in a non-solid state and cured or applied using a biocompatible adhesive. Said adhesives may include: pressure sensitive adhesives, polymeric tackifiers, hot-melt adhesives, curable adhesives, and repositionable adhesives such as microsphere pressure sensitive adhesives.

An advantage of the presently disclosed heat exchanger is that it can be produced more affordably than active heat exchangers. Active heat exchangers may cost approximately $2,500 on the market which likely arises from the manufacturing costs associated with the pump, cooling tank, and liquid channels and tubes.

These and other objects and advantages of the presently disclosed heat exchanger will become apparent to those of ordinary skill in the art after having read the following detailed description of the preferred embodiments which are illustrated in the various figures.

### BRIEF DESCRIPTION OF THE DRAWINGS

A more particular description of the heat exchanger briefly described above will be rendered by reference to specific embodiments which are illustrated in the appended drawings. Understanding that these drawings depict only typical embodiments of the heat exchanger and are not limiting of its scope, the heat exchanger will be described and explained with additional specificity and detail through the use of the accompanying drawings in which:

FIG. 1 shows a perspective, overhead view of a heat exchanger comprising an elastic bandage with elastomeric substrate and a non-adhering film.

FIG. 2 shows a side view of a heat exchanger comprising an elastic bandage wound up in a roll.

FIG. 3 illustrates a heat exchanger comprising an elastic bandage and non-adhering film wrapped around a knee joint.

FIG. 4 illustrates an embodiment of the disclosure comprising a tubular elastomeric substrate

FIG. 5 illustrates a cross-section view of another embodiment of the disclosure, where a hydrophobic polymer layer is coated by a hydrophilic layer.

### DETAILED DESCRIPTION

### Overview

One approach to the foregoing issues involves using standard elastic bandages, such as those commercially sold under the Ace brand name, submerged in water and then frozen to create a cold compress. However, such methods are impractical because the bandages stick together when frozen, making them difficult to unroll and apply. A disposable layer such as plastic wrap disposed between the layers may partially prevent sticking, but this method generates significant waste and is not reusable.

Disclosed herein is a heat exchanger which can be tightly wrapped around complex shapes such a human body to efficiently cool the complex shape and then can be easily unwrapped after usage. An advantage of the presently disclosed heat exchanger is that it can more efficiently decrease the temperature of a desired object than passive and active heat exchangers that are commonly known within the art.

As will be appreciated by one skilled in the art, the presently disclosed heat exchanger may be embodied as a heat exchanger made from a conformable, elastic bandage with an elastomeric substrate, with a non-adhering film of a temperature resistant biocompatible material applied on a portion of one side of the substrate.

In exemplary embodiments, the bandage is stored in a tightly rolled form, submerged in a biocompatible liquid, and placed in a freezing apparatus. Once the desired temperature is reached in the freezing apparatus, the non-adhering film facilitates the unwinding of the bandage from the roll. The bandage can then be wrapped around a desired object, such as a part of the human body, and secured using a fastening means. After lowering the object's temperature for the desired period, the self-adhering bandage can be more easily unwound due to the non-adhering film and provide a cryotherapeutic effect when applied to a body part.

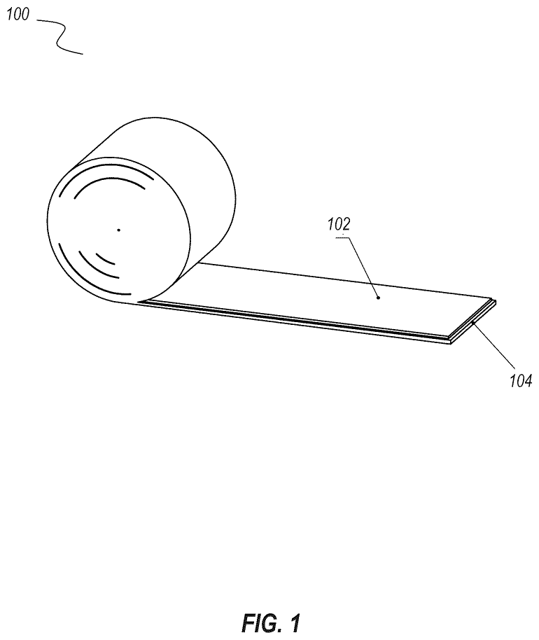

As illustrated in FIG. 1, a heat exchanger 100 made from said elastic bandage includes an elastomeric substrate 104 and a non-adhering film 102. In some embodiments, the film can be applied to one surface of the elastic bandage as two parallel stripes along the edges of the substrate, as a single central strip of silicone, or as a zig-zag pattern. Preferably, the film substantially coats the entirety of one surface of the elastic bandage.

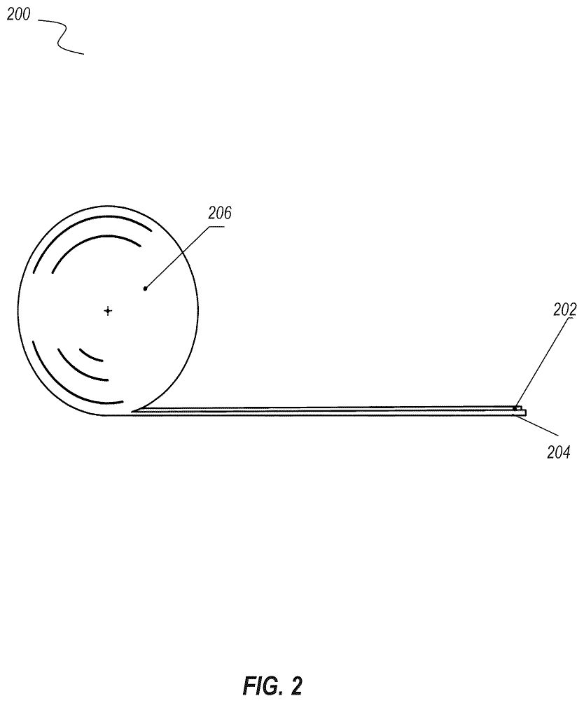

FIG. 2 illustrates a side perspective view of said elastic bandage partially unwound from a roll 206. As illustrated, the non-adhering film 202 is preferably disposed on the inside surface of the elastomeric substrate, such that the film prevents substantial portions of elastomeric substrate from touching one another.

Said elastic bandage can be produced through a long flat or tubular strip of elastomeric substrate that is used to support and compress a complex shape. In preferred embodiments, said elastic bandage may have various widths and lengths. In one preferred embodiment, the elastic bandage can be 4 inches wide by 5.5 yards long. In another embodiment, the elastic bandage can have dimensions of 4 inches wide by 11 yards long.

The elastomeric substrate 204 may preferably be comprised of a combination of synthetic and natural fibers including monofilament, multifilament, and natural staple fibers. The synthetic and natural fibers may be spun into thread and processed into woven or nonwoven materials, such as through meltblown extrusion, to form the elastomeric substrate. Synthetic fibers may be made from polyesters, polyamides, polypropylene polylactic acids (PLA), polyethylene terephthalates (PET), polyether-polyurea, styrene butadiene, copolymers such as elastane, styrene butadiene and polyisoprene, or polyurethane-polyurea copolymers. Natural fibers may be made from cotton, isoprene, polyisoprene, or regenerated cellulosic fibers such as rayon, acetate, and triacetate.

In some embodiments, said elastic bandage can include long stretch woven fabric that can stretch from 140-300% of their original length, helping provide constant pressure on a wrapped body part over a wide range of movement. Said elastic bandage can provide varying levels of compressive force between 1 to 70 mmHg to suit different therapeutic needs. Said elastic bandage can also include short-stretch woven fabric that can stretch from 30-90% of its original length. Preferred embodiments of the elastic bandage can have a high stretchability and low hysteresis, enabling reusability.

The non-adhering film can be adhered to the elastomeric substrate through extrusion in a non-solid state and cured or applied using a biocompatible adhesive. Said adhesives may include: pressure sensitive adhesives such as polyacrylates, polyvinylethers, and poly alpha-olefins; polymeric tackifiers such as natural rubber, styrene-isoprene block copolymer, silicone rubber, cis-polyisoprene, styrene butadiene, and cis-polybutadiene; hot-melt adhesives such as low-density polyethylene, ethylene-vinyl acetate copolymers, ethylene-ethyl acrylate copolymers, paraffin waxes, polyesters, polyamides, polypropylene, styrene-butadiene block copolymers and polyurethanes; curable adhesives such as silicones and urethanes; and repositionable adhesives such as microsphere pressure sensitive adhesives.

The thickness of the non-adhering film can range from 0.3 mm to 2 mm, or from 0.5 mm to 1 mm, or a range using any combination of the foregoing as endpoints. A thickness within such ranges balances durability, flexibility, and non-stick performance. For example, a film thinner than 0.3 mm may compromise structural integrity, making it prone to wear or tearing during repeated use and washing. Conversely, a film thicker than 2 mm may reduce the bandage's flexibility, hindering its ability to conform closely to complex body shapes, such as joints. The foregoing ranges provide an effective combination of resilience and elasticity, ensuring the bandage maintains its form and usability while effectively preventing adhesion when frozen.

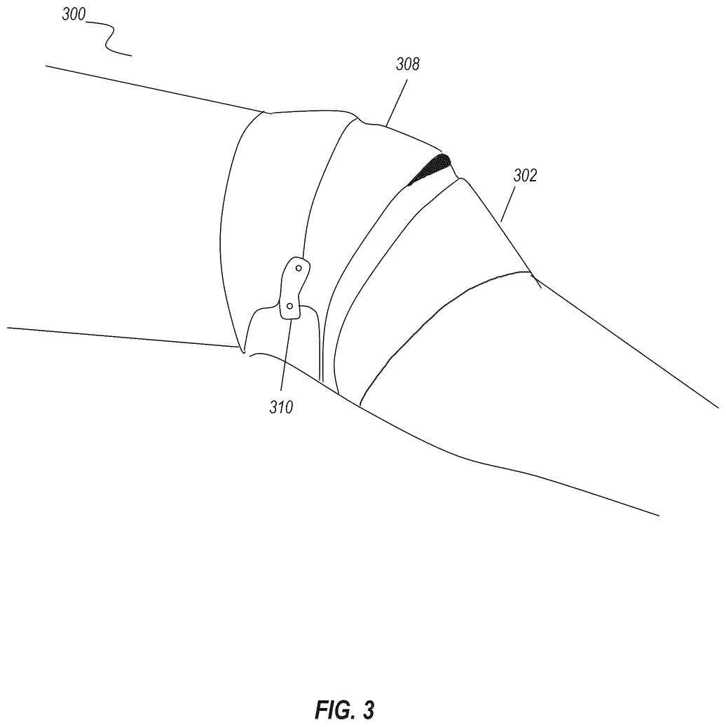

FIG. 3 illustrates a frozen elastic bandage wrapped around a knee joint 308. In preferred embodiments, the non-adhering film 302 faces away from the knee joint 308. The elastic bandage is secured by clip 310, but a person of ordinary skill in the art would appreciate that other fastening means could additionally or alternatively be utilized (e.g., hook and loop fastener, self-adhering components of the elastic bandage, etc.).

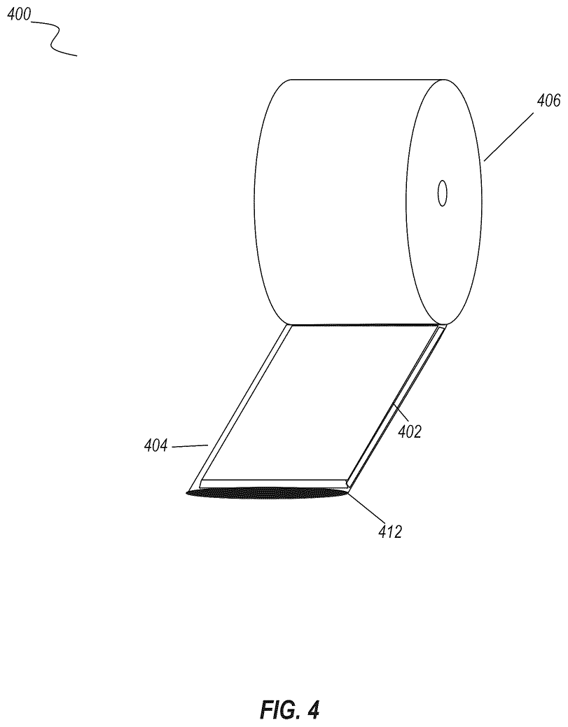

FIG. 4 illustrates an embodiment of the present disclosure where the elastic bandage is in the form of a roll 406, with the non-adhering film 402 is disposed on an inner surface of the elastomeric substrate 404. In this embodiment, the elastic bandage is configured to be tubular, defining a cavity 412 that extends longitudinally through the entire length of the elastic bandage.

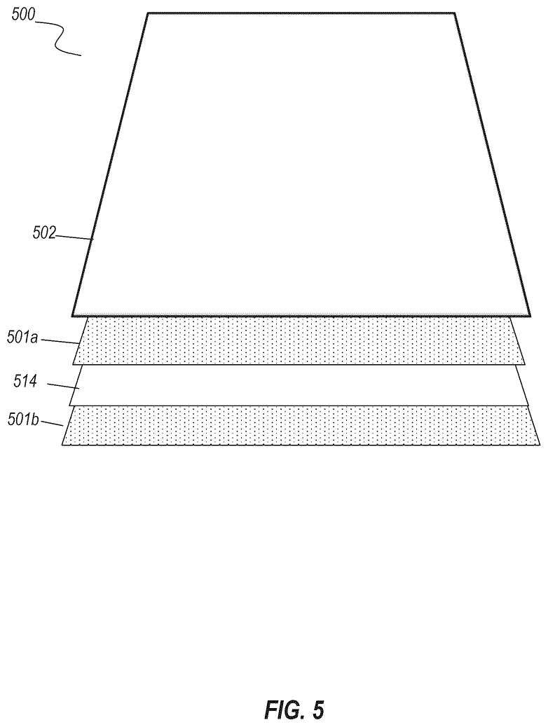

FIG. 5 illustrates an embodiment of the elastic bandage in which the elastomeric substrate of said elastic bandage can include hydrophilic material 501a and 501b to absorb biocompatible liquids. Hydrophilic fibers may preferably include cotton, cellulose, rayon, acetate, triacetate, other cellulose derivatives, nylon, or hydrogel polymeric materials. In some embodiments, hydrophilic fibers may coat a layer 514 of hydrophobic polymers like polyester, polyolefin, polyacrylonitrile and polyurethane to improve the wetting of these hydrophobic polymers. Preferably, hydrophilic coatings may be made from starches or hydrophilic polymers such as polyvinylpyrrolidone, poly-carboxyl acids, esters, salts and amides of poly(meth)acrylic acid, copolymers of poly (methyl vinyl ether/maleic anhydride), and polyglycols like polyethylene glycol. A non-adhering film 502 can lie on top of the hydrophilic coating layer 501a.

In use, the elastic bandage can be submerged in biocompatible liquids, which may include water, glycerol, ethanol, limonene, transcutol, ethyl acetate, polyethylene glycol, cremophor-EL (CEL), N-methyl-2-pyrrolidone, dimethyl acetamide, and/or ionic liquids such as derivatives from choline, carbohydrates, and/or amino acids. These biocompatible liquids may be used by themselves, in combination, or as solvents in solution with biocompatible solutes. Preferably, the biocompatible liquids have a freezing point sufficiently low to provide a cryotherapeutic effect when a saturated elastic bandage is applied to a body part.

In exemplary embodiments, the elastic bandage can be submerged in a biocompatible liquid while wound up in a roll, then while at least partially soaked with the biocompatible liquid, placed in a freezing apparatus (e.g., a common freezer). Advantageously, after reaching the desired temperature, the elastic bandage can be readily unwound because of the non-adhering film.

Illustrative embodiments of the presently disclosed heat exchanger may include the non-adhering film made from a biocompatible and temperature resistant material. The non-adhering film may be made from a hydrophobic film. Said hydrophobic films can be made from temperature resistant polyethylene, polyvinylidene chloride, polyurethane, and silicones such as polymerized siloxanes that can include octamethylcyclotetrasiloxane, hydroxy-terminated dimethyl siloxane, polydimethylsiloxane, methyltriacetoxysilane, and ethyltriacetoxysilane and can be reinforced with silica and metal oxides. In a preferred embodiment, the non-adhering film can reliably perform its intended function at temperatures between −48° C. to 177° C. For example, a non-adhering film comprising a 100% silicone that remains elastic at −48° C. to 149° C. is one presently preferred embodiment.

### Working Example

Different potential materials and configurations for the non-adhering film were tested by applying to elastic bandages and testing reusability, ability to be submerged in water and frozen, ability to remain elastic without cracking, and the ability to be unrolled in a frozen state. Tested materials included acrylic-latex blend, siliconized acrylic-latex, latex, silicon blend, 100% pure silicone, and polyethylene release film. These were tested with various application configurations including coating the full side of the substrate, two running beads along the substrate, a zig-zag pattern, and a single centered bead. The most effective material/configuration combination was 100% silicone, fully coating one side of the elastic bandage. This combination did not crack when stretched while frozen, remained adhered to the elastic bandage, and released when unrolled with minimal effort. While other combinations may have use in certain applications, they were more prone to cracking when stretched in a frozen state and/or were more difficult to unroll when frozen. The 100% silicone, fully coated example was therefore determined to be the most preferred.

What is claimed is:

1. A heat exchanger comprising:
an elastic bandage, comprising an elastomeric substrate with two opposing surfaces; and
a non-adhering film applied to at least a portion of at least one surface of the elastomeric substrate.

2. The heat exchanger of claim 1, wherein the elastic bandage is configured to be rolled into a cylindrical form.

3. The heat exchanger of claim 2, wherein the non-adhering film is disposed on an inner surface of the elastomeric substrate, the non-adhering film preventing substantial contact between overlapping layers of the elastomeric substrate when rolled into the cylindrical form.

4. The heat exchanger of claim 1, wherein the non-adhering film comprises a hydrophobic material that includes silicone, wherein the hydrophobic material comprises a thickness between 0.5 mm to 1 mm.

5. The heat exchanger of claim 1, wherein the elastomeric substrate comprises a blend of synthetic and natural fibers selected from monofilament, multifilament, and staple fibers.

6. The heat exchanger of claim 4, wherein the synthetic fibers comprise one or more of polyesters, polyamides, polypropylene, polylactic acids (PLA), polyethylene terephthalates (PET), polyether-polyurea copolymers, styrene butadiene copolymers, elastane, and polyisoprene.

7. The heat exchanger of claim 4, wherein the natural fibers comprise one or more of cotton, isoprene, polyisoprene, rayon, acetate, and triacetate.

8. The heat exchanger of claim 1, wherein the elastic bandage has a width of approximately 4 inches and a length of approximately 5.5 yards.

9. The heat exchanger of claim 1, wherein the elastic bandage has a width of approximately 4 inches and 11 yards.

10. The heat exchanger of claim 1, wherein the elastic bandage stretches to between 140% and 300% of its original length.

11. The heat exchanger of claim 1, wherein the elastic bandage stretches to between 30-90% of its original length.

12. The heat exchanger of claim 1, wherein the elastic bandage provides a compressive force ranging from 1 mmHg to 70 mmHg.

13. The heat exchanger of claim 1, wherein the elastomeric substrate includes hydrophilic fibers capable of absorbing biocompatible liquids.

14. The heat exchanger of claim 13, wherein the hydrophilic fibers coat hydrophobic polymers, the hydrophobic polymers comprising one or more of polyester, polyolefin, polyacrylonitrile, and polyurethane.

15. The heat exchanger of claim 12, wherein the hydrophilic fibers comprise one or more of starches, polyvinylpyrrolidone, polycarboxylic acids, esters, salts, amides of poly(meth)acrylic acid, copolymers of poly(methyl vinyl ether/maleic anhydride), and polyglycols.

16. The heat exchanger of claim 1, wherein the elastic bandage is configured to be tubular.

17. A method of manufacturing a heat exchanger, comprising:
forming an elastomeric substrate with a first surface and an opposing second surface;
applying a non-adhering film of a temperature-resistant, biocompatible material to a portion of a first surface of the elastomeric substrate; and
rolling the elastic bandage with the non-adhering film positioned to prevent contact between overlapping layers of the second surface.

18. The method of claim 17, wherein the non-adhering film is applied to entirely coat the first surface of the elastomeric substrate.

19. A method of using a heat exchanger, comprising:
providing an elastic bandage, wherein the elastic bandage comprises an elastomeric substrate with two opposing surfaces;
providing a non-adhering film applied to at least a portion of at least one surface of the elastomeric substrate;
submerging the elastic bandage in a biocompatible liquid and placing the elastic bandage in a freezing apparatus;
removing the elastic bandage from the freezing apparatus; and
wrapping the elastic bandage around a human body part and securing the elastic bandage with a fastening means.

20. The method of claim 19, wherein a bandage clip is used to secure the elastic bandage.
